(19) United States
(12) Patent Application Publication
(10) Pub. No.: US 2016/0116364 A1
(43) Pub. Date: Apr. 28, 2016

(54) Leak Test Apparatus and Method

(71) Applicant: AIR PRODUCTS AND CHEMICALS, INC., Allentown, PA (US)

(72) Inventors: Brian Thomas Vaccaro, Mertztown, PA (US); Jeffrey Michael Barna, Northampton, PA (US); David Charles Winchester, Walnutport, PA (US); David John Farese, Riegelsville, PA (US); Robert Kevin Wagner, Whitehall, PA (US); Brian Elwin Miller, Macungie, PA (US); Christopher Michael Albright, Allentown, PA (US); Lucas Anthony White, Laguna Niguel, CA (US)

(73) Assignee: AIR PRODUCTS AND CHEMICALS, INC., Allentown, PA (US)

(21) Appl. No.: 14/522,682

(22) Filed: Oct. 24, 2014

(51) Int. Cl. G01M 3/20; G01N 33/00

(52) U.S. Cl. CPC G01M 3/20; G01N 33/005

(57) ABSTRACT

A leak test apparatus for leak testing a tank including a gas supply system configured to provide to the tank a leak detection gas mixture including hydrogen, a test chamber having a cavity for sealably enclosing the tank such that dead space exists outside the tank and within the cavity, and a gas conduit configured to interconnect the gas supply system and the tank, a control system including a controller programmed to pressurize the tank with the leak detection mixture by flowing gas from the gas supply system through the gas conduit, and a leak detector to determine the presence or absence of a leak in the tank by measuring a concentration of hydrogen in the dead space.

### BACKGROUND

This application relates to a leak test apparatus and method that does not require helium, and in particular to a leak test apparatus and method that takes advantage of the small molecular size of hydrogen while providing appropriate safeguards against combustion or explosion.

Composite tanks (also known as composite overwrapped pressure vessels) are commonly used as fuel tanks for natural gas and hydrogen powered vehicles, as well as for storage of other potentially hazardous materials. Such tanks must be tested to verify the structural integrity of the tank. Conventionally, structural testing is performed using a hydro test, in which the tank is filled with liquid and then pressurized. An advantage of hydro testing is that in the event of a catastrophic failure or rupture of the tank, liquid does not expand in volume upon relief of pressure in the way that gas would expand, thereby significantly alleviating safety concerns from such a failure. Hydro testing is quite common practice, is safe, and equipment is readily available.

In addition, recent industry standards such as NGV2 now require tank manufacturers to perform leak testing to identify smaller/finer leaks. Leak testing such large vessels is challenging due to lack of equipment and methods. Present industry standards specify certain test requirements but do not provide much detail regarding how to perform the test or what equipment to use. Some present leak testing methods are targeted to specific portions of a tank where leaks are likely to occur, but are not capable of comprehensive leak testing of an entire large tank.

Helium is often used for leak testing, due to its small size and ability to find small leaks. However, helium is expensive due to limited supply. Also, composite tanks cannot typically hold vacuum pressures due to risk of buckling/collapse, so inboard leak testing is not an option. Therefore, it would be desirable to have an outbound leak test apparatus and method capable of safely leak testing large tanks.

### SUMMARY

A leak test apparatus and method are described herein in which a blend of hydrogen (H2) gas and an inert gas such as nitrogen (N2) gas is used to internally pressurize a tank to be leak tested. During the test, the tank is positioned in a test chamber, and the internal pressure is maintained in the tank for a predetermined time. Small leaks are detected by measuring an amount or an increase of hydrogen in the accumulation chamber.

Various embodiments of a leak test apparatus and method are described.

In one embodiment, a leak test apparatus is described for leak testing a tank. The apparatus includes a gas supply system configured to provide to the tank a leak detection gas mixture including hydrogen, a test chamber having a cavity for sealably enclosing the tank such that dead space exists outside the tank and within the cavity, and a gas conduit configured to interconnect the gas supply system and the tank, a control system including a controller programmed to pressurize the tank with the leak detection mixture by flowing gas from the gas supply system through the gas conduit, and a leak detector to determine the presence or absence of a leak in the tank by measuring a concentration of hydrogen in the dead space.

In one aspect of the leak test apparatus, the test chamber is changeable between an open configuration to enable insertion and removal of the tank and a closed configuration in which the tank is sealed within and surrounded by the cavity.

In another aspect of the leak test apparatus, the leak detection gas mixture further includes an inert gas, and the gas supply system includes a hydrogen valve for regulating flow of hydrogen into the tank and an inert gas valve for regulating flow of inert gas into the tank, including for purging and filling to a test pressure. In another aspect of the leak test apparatus, the gas supply system further includes a pump for boosting the pressure of the inert gas.

In another aspect of the leak test apparatus, the gas supply system further includes a vent valve for venting the leak detection mixture from the tank.

In another aspect of the leak test apparatus, the test chamber further includes at least one recirculating fan in the cavity for homogenizing gas in the cavity surrounding the tank.

In another aspect of the leak test apparatus, the test chamber further includes a safety relief device.

In another aspect of the leak test apparatus, the test chamber further includes a vent valve for exhausting gas from the cavity. In another aspect of the leak test apparatus, the test chamber further includes a fan configured to draw flow from the cavity through the test chamber vent valve. In another aspect of the leak test apparatus, the test chamber further includes a hydrogen safety sensor positioned within the cavity, and the controller is programmed to activate the fan and open the vent valve when the hydrogen safety sensor detects an unsafe concentration of hydrogen in the cavity.

In another aspect of the leak test apparatus, the leak detector includes a hydrogen sensor. In another aspect of the leak test apparatus, the leak detector further includes a sampling pump configured to draw a sample from the cavity to the hydrogen sensor.

In another aspect of the leak test apparatus, the leak detector further includes an indicator to indicate detection of a leak.

In another aspect of the leak test apparatus, the test chamber further includes filler material positioned outside the tank and within the cavity to reduce the volume of the dead space. In another aspect of the leak test apparatus, the filler material includes an inflatable bladder, water, or both.

In another embodiment, a method of leak testing a tank is described. The method includes enclosing the tank in a cavity of a test chamber such that a dead space is present outside the tank and within the cavity, purging the tank with an inert gas to reduce the concentration of O2 to a safe level, flowing hydrogen into the tank to achieve a fill pressure, flowing an inert gas into the tank to increase the pressure in the tank from the fill pressure to a test pressure and to dilute the concentration of hydrogen to a test concentration, holding pressure in the tank for a predetermined hold time, measuring the concentration of hydrogen in the dead space, and detecting a leak if the concentration of hydrogen in the dead space exceeds a predetermined threshold.

In one aspect of the method, the inert gas is nitrogen.

In another aspect of the method, the test pressure is from about 3600 PSIG to about 5000 PSIG.

In another aspect of the method, the test concentration of hydrogen is from about 4% to about 6%.

In another aspect of the method, the predetermined hold time is from about 5 minutes to about 60 minutes.

In another aspect of the method, the predetermined threshold is from about 1 ppm to about 100 ppm.

In yet another embodiment, a leak test apparatus for leak testing a tank is described. The apparatus includes a test chamber having a cavity and a gas conduit configured to provide a leak detection gas mixture to the tank, the test chamber being changeable between an open configuration to enable insertion and removal of the tank and a closed configuration in which the tank is sealed within the cavity such that dead space exists outside the tank and within the cavity, the leak detection gas mixture including hydrogen and nitrogen. The apparatus further includes a gas supply system including a low pressure nitrogen valve to provide purge gas to the tank via the gas conduit, an hydrogen valve to provide hydrogen to the tank via the gas conduit, a booster pump and high pressure nitrogen valve to provide nitrogen to the tank and to pressurize the tank to a test pressure via the gas conduit, and a vent valve for venting the tank via the gas conduit. The apparatus further includes a control system including a controller programmed to purge the tank by actuating the low pressure nitrogen valve, to provide hydrogen to the tank by actuating the hydrogen valve, to pressurize the tank to a test pressure with the leak detection gas mixture by providing nitrogen to the tank by actuating the booster pump and high pressure nitrogen valve, and to vent the tank via the gas conduit. The apparatus further includes a leak detector configured to determine the presence or absence of a leak in the tank by measuring a concentration of hydrogen in the cavity dead space, the leak detector including a hydrogen sensor, a sampling pump configured to draw a sample form the cavity to the hydrogen sensor, and an indicator to indicate detection of a leak.

In yet another embodiment, a method of leak testing a tank is described. The method includes enclosing the tank in a cavity of a test chamber such that a dead space is present outside the tank and within the cavity, purging the tank with nitrogen to reduce the concentration of O2 to less than or equal to about 1%, flowing hydrogen into the tank to achieve a fill pressure, flowing nitrogen into the tank to increase the pressure in the tank from the fill pressure to a test pressure and to dilute the concentration of hydrogen to a test concentration, holding pressure in the tank for a predetermined hold time, measuring the concentration of hydrogen in the dead space, and detecting a leak if the concentration of hydrogen in the dead space exceeds a predetermined threshold, wherein the test parameters include, either separately or in any combination with each other, the test pressure being from about 3600 PSIG to about 5000 PSIG, the test concentration of hydrogen being from about 4% to about 6%, the predetermined hold time being from about 5 minutes to about 60 minutes, and the predetermined threshold being from about 1 ppm to about 100 ppm.

It is understood that any of the foregoing aspects may be used separately or in combination with any one or more of the other foregoing aspects.

Other aspects of the invention are described below.

### BRIEF DESCRIPTION OF THE DRAWINGS

FIG. 1 is a schematic showing an embodiment of a leak test apparatus.

FIG. 2 is a flow chart showing an embodiment of a leak test method.

FIG. 3 is a schematic showing another embodiment of a leak test apparatus.

FIG. 4 is a schematic showing an embodiment of a leak test system including stations for leak testing multiple tanks of different sizes.

### DETAILED DESCRIPTION

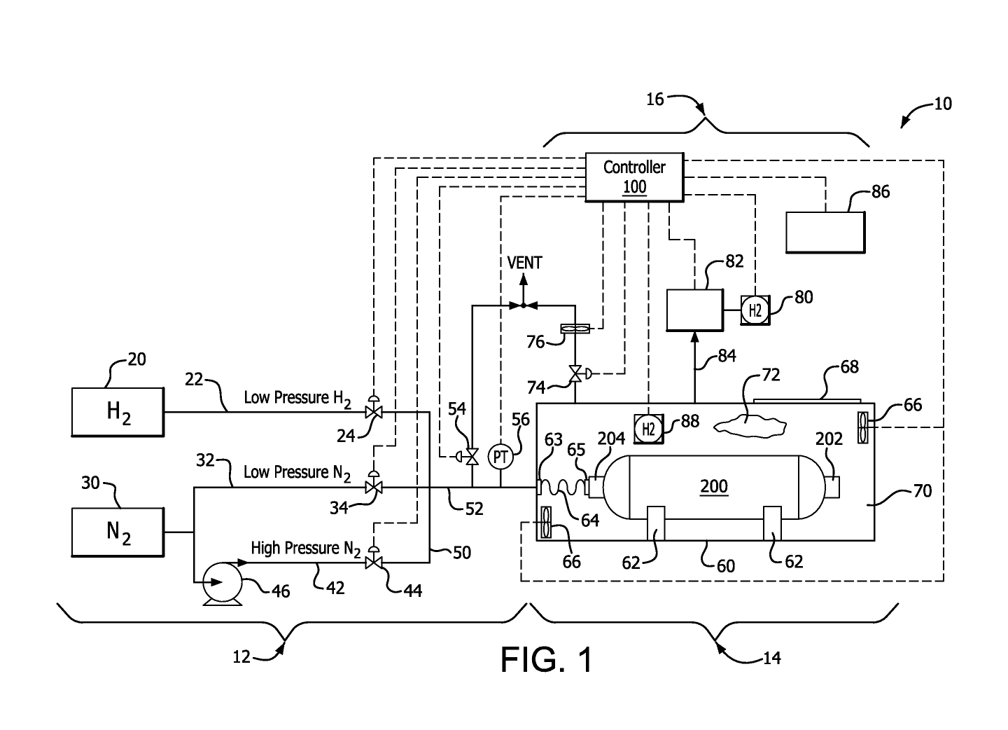

FIG. 1 depicts an embodiment of a leak test apparatus 10. The apparatus 10 includes a gas supply system 12, a test chamber 14, and a control system 16.

The gas supply system 12 is configured to selectively supply low pressure hydrogen, low pressure nitrogen or other inert gas, and high pressure nitrogen or other inert gas, separately or in combination, to the test chamber 14. As used herein, the term “inert” gas means any gas that is essentially non-reactive with hydrogen at ambient temperatures. In the depicted embodiment, the gas supply system 12 includes a hydrogen source 20 that feeds a hydrogen conduit 22, and a nitrogen source 30 that feeds a low pressure nitrogen conduit 32 and a high pressure nitrogen conduit 42. A gas manifold 50 joins the downstream ends of the hydrogen conduit 22, the low pressure nitrogen conduit 32, and the high pressure nitrogen conduit 42 into a single gas conduit 52.

A hydrogen control valve 24 is positioned in the hydrogen conduit 22 for regulating the flow of hydrogen through the hydrogen conduit 22. A low pressure nitrogen control valve 34 is positioned in the low pressure nitrogen conduit 32 for regulating the flow of nitrogen through the low pressure nitrogen conduit 32. A booster pump 46 is positioned in the high pressure nitrogen conduit 42 for increasing the pressure of the nitrogen supplied by the nitrogen source 30, and a high pressure nitrogen control valve 44 is positioned in the high pressure nitrogen conduit 42 downstream of the booster pump 46 for regulating the flow of nitrogen through the high pressure nitrogen conduit 42. Each of the control valves 24, 34, and 44 is independently controlled by a controller 100 in the control system 16.

The test chamber 14 includes an enclosure 60 having an internal cavity 70 that is configured to receive and enclose a tank 200. The tank includes a first boss port 202 and a second boss port 204. The enclosure 60 is changeable from a first or open position in which the tank 200 can be inserted into or removed from the cavity 70 and a second or closed position in which the tank 200 is sealed within the cavity 70. For example, the enclosure 60 may be constructed in a clamshell or coffin configuration, hinged on one edge, with a perimeter seal between two halves or portions of the enclosure 60.

In the closed position of the enclosure 60, when the tank 200 is sealed within the cavity 70, a dead space exists between the tank 200 and the enclosure 60 that is filled with air. As discussed below in detail, leak detection will be performed by measuring the concentration of hydrogen in the dead space. Therefore, it is preferable to reduce or minimize the dead space volume (i.e., the sampled space) to thereby increase the test accuracy and/or quicken the response time of leak testing. In other words, a smaller dead space volume may enable one or more of higher detection thresholds, faster leak detection, accurate detection of leaks in smaller tanks for the same size enclosure, and detection of smaller leaks. In one example, for a given size tank and a given size leak, decreasing the dead space volume will cause the measured hydrogen concentration in the dead space to reach a detection threshold sooner. In another example, for a given leak detection time and leak size, a higher threshold (i.e., a less sensitive detector) can be used for detection when the dead space volume is smaller.

A filler material 72 may be used to reduce the dead space volume between the tank 200 and the enclosure 60. Using filler material also enables the enclosure 60 to accommodate various size tanks 200 and to adjust the dead space volume for each tank to maintain a reasonable response time and hydrogen concentration threshold. In one embodiment the filler material is an inflatable bladder. In another embodiment, the filler material is water. However, it is understood that the filler material may be any material or structural element, or combinations of materials and/or structural elements, that are capable of filling a portion of the dead space and do not affect the hydrogen concentration of the dead space (i.e., the filler material should not emit or absorb hydrogen).

The enclosure may include one or more cradles 62, as shown, to support the tank 200 within the cavity 70. A fitting 63 mounted in a wall of the enclosure 60 is configured to mate with the gas conduit 52 of the gas manifold 52. Another gas conduit 64 is connected to the fitting 63 and extends into the cavity 70 for connection with a fitting 65 that is configured to mate with a gas inlet in the boss port 204 on the tank 200. In one embodiment, the gas conduit 64 is a flexible conduit or hose to accommodate connection to various size tanks 200 that may be leak tested in the test chamber 14.

A vent valve 54 is mounted to the gas conduit 52 in the gas manifold 50 for exhausting pressurized gas from the tank 200 during pre-test purging of the tank 200 and after the completion of a leak test. A pressure transmitter 56 mounted to the gas conduit 52 is used to measure and transmit the fill pressure of the tank 200 as it is being filled and while pressure is being maintained during a leak test. The pressure transmitter 56 sends a pressure signal to the controller 100, which uses that pressure signal to modulate the control valves 24, 34, and 44, as well as the vent valve 54, depending on the desired pressure setpoint and the state of operation of a leak test.

One or more recirculating fans 66 may be mounted within the cavity 70 of the enclosure 60, to improve homogeneity of the gas in the dead space surrounding the tank 200. In the depicted embodiment, two recirculating fans 66 are shown, it being understood that a different number of fans 66 may be used depending on the size of the enclosure 60 and the size of the tank 200, and it being further understood that not all fans 66 need be operated at the same time or at the same speed. For safety, a blow-out plate 68 or other pressure relief device is mounted in the wall of the enclosure 60 to prevent overpressurization of the enclosure 60.

A vent valve 74 is mounted to the enclosure 60 for venting the cavity 70, and an exhaust fan 76 may be provided downstream of the vent valve 74 to accelerate the exhausting of gas from the cavity 70, particularly in the event of a high hydrogen level is detected in the dead space between the tank 200 and the enclosure 60.

The control system 16 includes the controller 100, as well as a hydrogen sensor 80 for detecting the concentration of hydrogen in the dead space in the cavity 70 surrounding the tank 200 being leak tested. Preferably, a sampling pump 82 draws a gaseous sample through a conduit 84 from the cavity 70 into the hydrogen sensor 80, which then determines whether the hydrogen concentration is at or above a threshold hydrogen level that indicates a leak in the tank 200. The sampling pump 82 is controlled by the controller 100, and may be operated to draw a continuous sample or periodic samples, depending on the desired detection result and methodology. An indicator, such as a beacon, a light, or an audible alarm, may be connected to the hydrogen sensor 80 to indicate the presence of a leak in the tank 200, if the hydrogen sensor 80 detects a hydrogen concentration above the threshold level. The controller 100 is operatively connected to be able to independently regulate each of the control valves 24,34, and 44, the vent valves 54 and 74, the sampling pump 82, the hydrogen sensor 80, optionally the indicator 86, the exhaust fan 76, and the recirculating fans 66, and to receive signal inputs from the pressure transducer 56 and the hydrogen sensor 80.

The apparatus 10 uses a blend of hydrogen gas and nitrogen gas to pressurize a tank 200 in the enclosure 60, and holds the pressure for a predetermined time to check for small leaks in the tank 200 using a hydrogen leak detector. In one embodiment, the tank 200 is pressurized with a gas mixture of hydrogen and nitrogen (or other inert gas) having a hydrogen concentration about at the hydrogen lower explosive limit (LEL) of 4%. In another embodiment, the tank 200 is pressurized with a gas mixture of hydrogen and nitrogen (or other inert gas) having a hydrogen concentration somewhat above the LEL, for example up to about 5% or up to about 6%. A higher hydrogen concentration enables faster leak detection. It is understood that any leak in the tank 200 will reduce the hydrogen concentration in the tank 200, and the concentration of leaked hydrogen outside the tank 200 and inside the enclosure 60 will be at the ppm level in any event, so the risk of having a hydrogen concentration above the LEL in the tank 200 is minimal. In addition the purge and test procedures ensure that the tank 200 has low levels of oxygen prior to being filled with the hydrogen-containing gas mixture. Further, no sources of ignition are present in the tank 200.

The enclosure 60 further includes a hydrogen safety sensor 88 positioned in the dead space of the cavity 70 outside the tank 200. In contrast to the hydrogen sensor 80, the hydrogen safety sensor 88 is set to alarm if a potentially unsafe level of hydrogen is detected in the dead space. The hydrogen safety sensor 88 may be set to alarm at a threshold of less than 4% hydrogen in the cavity 70. For example, the hydrogen safety sensor 88 may be set to a threshold of about 1% or about 2% or about 3% hydrogen in the dead space. The hydrogen safety sensor 88 sends a signal to the controller 100 if the hydrogen in the dead space reaches or exceeds the alarm threshold. In response to such a signal, the controller 100 acts to decrease the hydrogen concentration in the cavity 70 and prevent further hydrogen accumulation in the cavity 70. These actions include one or more of: opening the vent valve 74 and activating the exhaust fan 76 to quickly evacuate the cavity 70, and stopping flow of hydrogen and/or nitrogen into the vessel 200 and opening the vent valve 54 to vent the hydrogen-containing gas mixture from the vessel 200.

The hydrogen source 20 can be a low pressure hydrogen source. In one embodiment, the hydrogen source can deliver hydrogen up to about 200 PSIG. The nitrogen source 30 can be a low pressure nitrogen source. In one embodiment, the nitrogen source 30 can deliver nitrogen up to about 200 PSIG, and in another embodiment at up to about 300 PSIG. The nitrogen booster pump 46 is capable of delivering nitrogen at much higher pressures desirable for leak testing. The apparatus 10 may be configured to leak test a tank at any desired pressure, depending in part on the rated pressure of the tank. The apparatus 10 will typically be operated using a test pressure from about 2500 PSIG to about 6000 PSIG, preferably from about 3000 PSIG to about 5500 PSIG, and more preferably from about 3600 PSIG to about 5000 PSIG. In one embodiment, nitrogen is delivered at a pressure of at least about 3600 PSIG and in another embodiment up to about 5000 PSIG.

The enclosure 60 can be configured to accommodate any size tank, and in one embodiment is configured to accommodate common sizes of composite tanks being tested (i.e., tanks typically from 18″ to 26″ in diameter and from 60″ to 144″ in length). As described above, filler material or spacer pieces may be used to occupy unused volume or dead space in the cavity 70, depending on size of the tank 200 being tested, to minimize the air volume in the cavity 70. This could also be accomplished by using an inflatable bladder or filling the cavity dead space with water.

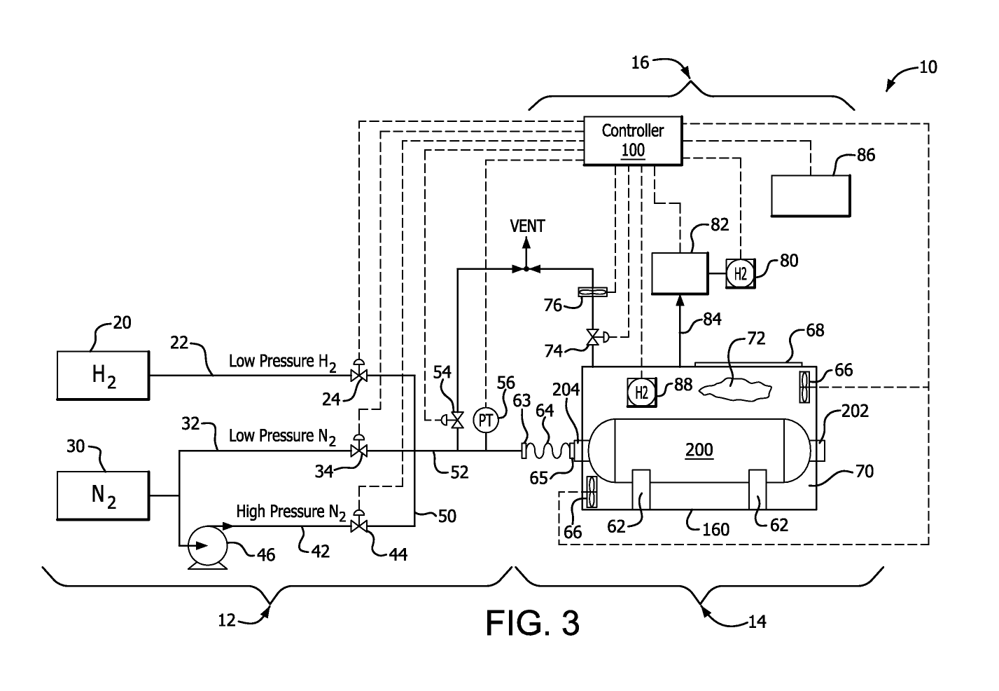

FIG. 3 depicts another embodiment of a leak detection apparatus 10. As indicate by the like reference numerals, this embodiment is nearly identical to the embodiment of FIG. 1, except for the boundaries of the sealed enclosure 160 in FIG. 3 relative to the tank 200 (as compared with the boundaries of the sealed enclosure 60 in FIG. 1 relative to the tank 200). The features common between the two embodiments will not be described again; instead the following description addresses the differences between the embodiments of FIG. 3 and FIG. 1.

As shown in FIG. 3, the boss ports 202 and 204 protrude outside the ends of the enclosure 160, and the enclosure 160 is sealed around the boss ports 202, 204. There are several benefits for this arrangement. Because the boss ports 202, 204 have a small diameter relative to the tank 200 and extend axially, positioning the boss ports 202, 204 within the enclosure 60 significantly increases the dead space that either must be filled with filler material 72 or must be accounted for as part of the sampled volume when detecting leak. Further, the boss ports 202, 204, as well as the fittings 63, 65 and the hose 64, are the most likely source of leaks in the system, so by positioning these elements outside the enclosure 160, a leak test can be focused solely on the integrity of the tank 200. Note that an additional hydrogen safety sensor (not shown, but similar to the hydrogen safety sensor 88) can be positioned outside the enclosure 160 to detect any substantial leaks that may occur in the boss ports 202, 204, the fittings 63, 65, and/or the hose 64.

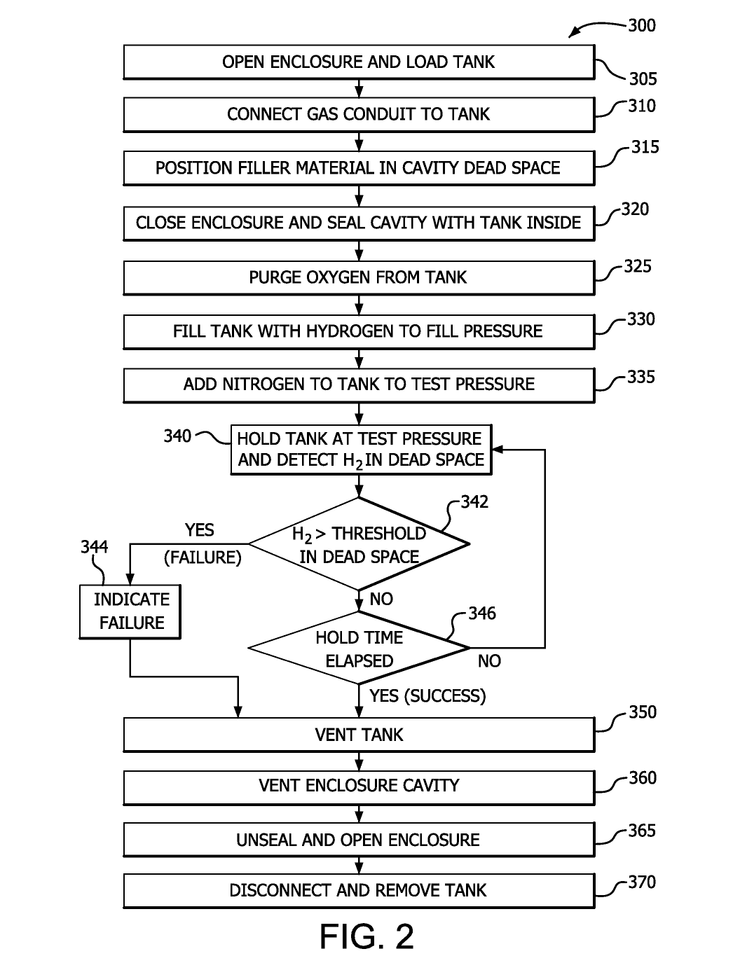

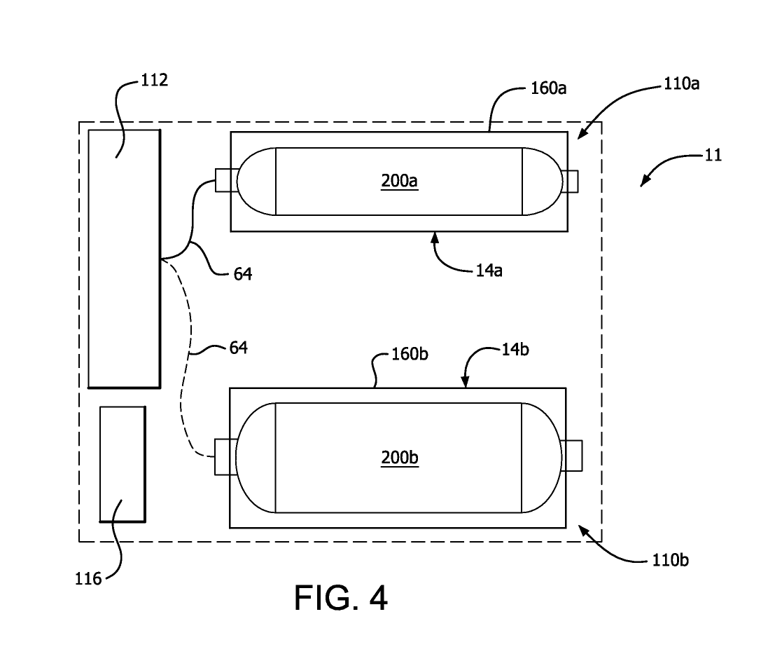

FIG. 4 shows a leak test system 11 configured with multiple test stations 110. As depicted, the system 11 has two test stations 110a and 110b, it being understood that any number of test stations 110 could be supported. A common gas supply system 112 performs all the functions needed to supply the test stations 110, and a common control system 116 performs all the functions needed to control leak testing and safety for the test stations 110, as described above with regard to the gas supply system 12 and the control system 16 in the analogous single-tank leak test apparatus 10. Each test station 110 includes its own test chamber 14 and enclosure 160, and can be connected via a hose 64 to the common gas supply system 112. A primary advantage of having multiple test stations 110a, 110b is that the separate enclosures 160a, 160b can be sized to accommodate differently sized tanks with minimal dead space, thereby enhancing test accuracy and/or decreasing test times, as discussed in more detail above, and may avoid the need for any filler material. Although the depicted leak test system 11 indicates that only one test chamber 14 can be connected at a time to the common gas supply system 112, the leak test system 11 could alternately be arranged, and the common gas supply system 112 and common control system 116 configured, to conduct multiple leak tests in parallel. One embodiment of an operation sequence 300 for leak testing a tank 200 is shown in FIG. 2. Other sequences may be used with equal effectiveness, as would be understood by a person of ordinary skill in the art.

In step 305, the enclosure 60 is placed in the open position and a tank 200 is loaded into the cavity 70. Loading of the tank 200 may be done manually or by an automated process. In step 310, the conduit 64, via the fitting 65, is connected to the tank 200 to enable the tank to be filled with a test gas. In step 315, if desired or necessary, filler material 72 is positioned in the dead space in the cavity 70. It is understood that steps 310 and 315 may be interchanged in order without negative impact. In step 320, the enclosure 60 is placed in the closed position and the cavity 70 is sealed.

In step 325, the controller 100 opens the low pressure nitrogen control valve 34 to flow low pressure nitrogen into the tank 200 for purging the tank of oxygen. The flow rate and time of nitrogen purge is set so as to reduce the oxygen concentration in the tank 200 to a safe level. As used herein, a “safe level” is an oxygen concentration insufficient to support ignition and/or combustion of a hydrogen-containing gas with up to about 4% or up to about 5% or up to about 6% hydrogen in an inert gas. In one embodiment, a safe level of oxygen concentration means less than or equal to about 1%. This calculation can be performed as a volumetric dilution based on the size of the tank 200, which may be inputted into the controller 100. For example, in one embodiment, since oxygen is about 21% of atmospheric air, about 20 tank volumes of nitrogen is flowed into the tank 200. This could be done by filling the tank 200 with nitrogen up to about 300 PSIG, as measured by the pressure transducer 56, and then venting the tank 20 through the vent valve 54 back to about atmospheric pressure (0 PSIG or 14.7 PSIA), or by twice successively filling the tank 200 with nitrogen up to about 150 PSIG and then venting the tank 200 through the vent valve 54 back to about atmospheric pressure, or another approach with a different number of fillings and ventings. In step 330, once the oxygen concentration in the tank 200 has been diluted to less than or equal to about 1%, the controller closes the control valve 34 and then opens the vent valve 54 to vent the tank 200 back to about atmospheric pressure containing a mixture of less than or equal to about 1% oxygen in primary nitrogen.

In step 330, the controller 100 opens the hydrogen control valve 24 to flow hydrogen into the tank 200 to achieve a desired fill pressure. In one embodiment, the tank 200 is filled to a fill pressure of about 130 PSIG, as measured by the pressure transducer 56, at which point the control valve 24 is closed. In step 335, the controller 100 activates the booster pump 46 and opens the high pressure nitrogen control valve 44 to flow nitrogen into the tank 200. Nitrogen is added to the tank 200 until the a desired test pressure is reached and the hydrogen concentration in the tank 200 is below the LEL. In one embodiment, the tank 200 is filled to a test pressure of about 3600 PSIG (about 3615 PSIA). Based on a hydrogen fill pressure of about 130 PSIG (about 145 PSIA), the final hydrogen concentration would be about 4%, or at about the LEL threshold. As noted above, in other embodiments, the tank 200 may be filled by a combination of hydrogen (to the fill pressure) and nitrogen (to the test pressure) to achieve a hydrogen concentration that is higher than the LEL, such as up to about 5% or up to about 6%. For example, to achieve a hydrogen concentration of about 6%, hydrogen would be filled into the tank to a fill pressure of about 202 PSIG, followed by nitrogen up to a test pressure of about 3600 PSIG. It is understood that if a higher test pressure is desired, then a correspondingly higher hydrogen fill pressure can be used to achieve about the same final hydrogen concentration. It is preferable to use a hydrogen concentration as high as safely possible. For example, if a test pressure of 5000 PSIG is desired, a hydrogen fill pressure of about 185 PSIG can be used to achieve a test hydrogen concentration of about 4%, or a hydrogen fill pressure of about 285 PSIG can be used to achieve a test hydrogen concentration of about 6%, followed by the addition of high pressure nitrogen to boost the test pressure to about 5000 PSIG.

In step 340, pressure is maintained in the tank 200 and the concentration of hydrogen in the dead space 70 is detected, continuously or periodically, by the hydrogen sensor 80. During this hold time, the control valves 24, 34, and 44 are all closed, the tank vent valve 54 is closed, and the cavity vent valve 74 is closed. Continuously or periodically during the hold time, the sampling pump 82 is activated to draw gas from the dead space in the cavity 70 to the hydrogen sensor 80, and the hydrogen concentration in the dead space is detected. If the tank 200 is leaking, hydrogen will escape from the tank 200 via the leak into the dead space of the cavity 70, and the hydrogen concentration in the dead space will increase. If the leak is sufficiently large, the hydrogen concentration in the dead space will increase to a detectable level above a threshold level during the hold time. The threshold level will depend on the dead space volume and the size of the tank 200, as well as the test pressure, all of which can be readily calculated by a person skilled in the art.

In step 342, if the hydrogen sensor 80 detects a hydrogen concentration of less than the threshold level, the process proceeds to step 346. In step 346, if the predetermined hold time has elapsed and the hydrogen sensor 80 does not detect a hydrogen concentration at or above the threshold level, then the tank 200 is deemed to have successfully passed the leak test, and operation proceeds to step 350. In step 346, if the predetermined time has not elapsed, operation returns to step 340 to continue retesting until the hold time elapses (testing loop).

In step 342, if the hydrogen sensor 80 detects a hydrogen concentration of equal to or greater than the threshold level during the hold time, the tank 200 is deemed to have failed the leak test. In step 344, the indicator 86 is activated to indicate a leak and thus failure of the test. Failure of the leak test concludes the testing loop and sends operation to step 350.

The hydrogen sensor 80 may be set to have a threshold from about 1 ppm to about 100 ppm, preferably from about 2 ppm to about 50 ppm, and more preferably from about 5 ppm to about 25 ppm, depending upon the tank size, test pressure, and dead space volume, as well as the hold time. Similarly, the hold time may be set to be from about 5 minutes to about 60 minutes, preferably from about 10 minutes to 45 minutes, and more preferably from about 15 minutes to 30 minutes, again depending upon the tank size, test pressure, and dead space volume, as well as the hydrogen detection threshold. In one exemplary embodiment, the hydrogen sensor 80 is set to a threshold of 5 ppm and the hold time is set to 30 minutes, and the apparatus is capable of detecting a leak rate on the order of 0.005 cubic centimeters per second (cc/sec). In other embodiments, other combinations of thresholds and hold times can be used to adjust the minimum detectable leak rate. Elapse of the hold time and successful completion of the leak test concludes step 345 and sends operation to step 350.

At any time concurrently with or after step 325, the controller 100 ensures that vent valve 74 is closed. Similarly, at any time concurrently with or after step 325, the controller 100 can turn on one or more of the recirculating fans 66. The recirculating fans 66 can remain active, or can operate intermittently or selectively, through steps 330, 335, 340, and 345.

In step 355, the tank 200 is vented by opening the vent valve 54. Venting of the tank 200 continues until the pressure transducer 56 detects a pressure of about atmospheric pressure. Because of the hydrogen content in the gas being vented, the vent outlet is outside and preferably at a safe distance above the ground. In step 360, the cavity 70 is vented by opening the cavity vent valve 74, and if deemed necessary, by activating the exhaust fan 76. The exhaust fan 76 is also helpful to evacuate even small amounts of hydrogen from the dead space in the cavity 70 so as to avoid any contamination (i.e., false positives) in each subsequent test of another tank after a previous tank (with or without a detected leak) has been removed. Further, if a large leak was detected, and in particular a leak sufficiently large to reach the detection threshold of the hydrogen safety sensor 88, then the exhaust fan 76 is activated for safety purposes to quickly vent the cavity 70.

In step 365, the enclosure 60 is unsealed and moved from the closed position to the open position. In step 370, the gas conduit 64 is disconnected from the tank 200 and the tank 200 is removed from the enclosure 60.

The apparatus 10 enables tank manufacturers, and in particular composite tank manufactures, to safely perform leak testing with hydrogen gas, which has a small molecular size similar to that of helium (He), but is much less expensive and readily available. In particular, the apparatus 10 enables detection of low leak rates (small leaks) in large tanks of various sizes.

In addition, the leak test performed by the apparatus 10 and corresponding method described herein is superior to a conventional pressure decay test for at least two reasons. First, the method described herein is not susceptible to temperature changes (e.g., due to gas heating during tank fill and due to ambient fluctuations during test hold), but rather measures only hydrogen leakage out of a tank. Second, pressure indicating instruments with the resolution/accuracy required to detect such small leak rates (i.e., changes in pressure) at such high test pressures are generally not available, so the present apparatus and method can detect leaks smaller than currently available technology used in convention pressure decay testing.

The present invention is not to be limited in scope by the specific aspects or embodiments disclosed in the examples which are intended as illustrations of a few aspects of the invention and any embodiments that are functionally equivalent are within the scope of this invention. Various modifications of the invention in addition to those shown and described herein will become apparent to those skilled in the art and are intended to fall within the scope of the appended claims.

1. A leak test apparatus for leak testing a tank, comprising:
a gas supply system configured to provide to the tank a leak detection gas mixture including hydrogen;
a test chamber having a cavity for sealably enclosing the tank such that dead space exists outside the tank and within the cavity, and a gas conduit configured to interconnect the gas supply system and the tank;
a control system including a controller programmed to pressurize the tank with the leak detection mixture by flowing gas from the gas supply system through the gas conduit; and
a leak detector to determine the presence or absence of a leak in the tank by measuring a concentration of hydrogen in the dead space.

2. The leak test apparatus of claim 1, the test chamber being changeable between an open configuration to enable insertion and removal of the tank and a closed configuration in which the tank is sealed within and surrounded by the cavity.

3. The leak test apparatus of claim 1,
the leak detection gas mixture further including an inert gas; and
the gas supply system including a hydrogen valve for regulating flow of hydrogen into the tank and an inert gas valve for regulating flow of inert gas into the tank.

4. The leak test apparatus of claim 3, the gas supply system further including a pump for boosting the pressure of the inert gas.

5. The leak test apparatus of claim 1, the gas supply system further including a vent valve for venting the leak detection mixture from the tank.

6. The leak test apparatus of claim 1, the test chamber further including at least one recirculating fan in the cavity for homogenizing gas in the cavity surrounding the tank.

7. The leak test apparatus of claim 1, the test chamber further including a safety relief device.

8. The leak test apparatus of claim 1, the test chamber further including a vent valve for exhausting gas from the cavity.

9. The leak test apparatus of claim 8, the test chamber further including a fan configured to draw flow from the cavity through the test chamber vent valve.

10. The leak test apparatus of claim 9, the test chamber further including a hydrogen safety sensor positioned within the cavity, wherein the controller is programmed to activate the fan and open the vent valve when the hydrogen safety sensor detects an unsafe concentration of hydrogen in the cavity.

11. The leak test apparatus of claim 1, the leak detector including a hydrogen sensor.

12. The leak test apparatus of claim 11, the leak detector further including a sampling pump configured to draw a sample from the cavity to the hydrogen sensor.

13. The leak test apparatus of claim 1, the leak detector further including an indicator to indicate detection of a leak.

14. The leak test apparatus of claim 1, the test chamber further including filler material positioned outside the tank and within the cavity to reduce the volume of the dead space.

15. The leak test apparatus of claim 14, wherein the filler material is selected from the group consisting of: an inflatable bladder, water, and combinations thereof

16. A method of leak testing a tank, comprising:
enclosing the tank in a cavity of a test chamber such that a dead space is present outside the tank and within the cavity;
purging the tank with an inert gas to reduce the concentration of O2 to a safe level;
flowing hydrogen into the tank to achieve a fill pressure;
flowing an inert gas into the tank to increase the pressure in the tank from the fill pressure to a test pressure and to dilute the concentration of hydrogen to a test concentration;
holding pressure in the tank for a predetermined hold time;
measuring the concentration of hydrogen in the dead space; and
detecting a leak if the concentration of hydrogen in the dead space exceeds a predetermined threshold.

17. The method of claim 16, wherein the test pressure is from about 3600 PSIG to about 5000 PSIG.

18. The method of claim 16, wherein test concentration of hydrogen is from about 4% to about 6%.

19. The method of claim 16, wherein the predetermined hold time is from about 5 minutes to about 60 minutes.

20. The method of claim 16, wherein the predetermined threshold is from about 1 ppm to about 100 ppm.
